(19) United States
(12) Patent Application Publication
(10) Pub. No.: US 2023/0024539 A1
(43) Pub. Date: Jan. 26, 2023

(54) KIT FOR DETECTING NUCLEIC ACID AND METHOD OF PREPARING THE SAME

(71) Applicant: RESEARCH & BUSINESS FOUNDATION SUNGKYUNKWAN UNIVERSITY, Suwon-si (KR)

(72) Inventors: Jinkee LEE, Suwon-si (KR); Youngkwang MOON, Suwon-si (KR); Hyeokgyun MOON, Suwon-si (KR); Jung Heon LEE, Suwon-si (KR); Junhyuck CHANG, Suwon-si (KR)

(73) Assignee: RESEARCH & BUSINESS FOUNDATION SUNGKYUNKWAN UNIVERSITY, Suwon-si (KR)

(21) Appl. No.: 17/858,326

(22) Filed: Jul. 6, 2022

(30) Foreign Application Priority Data
Jul. 6, 2021 (KR) 10-2021-0088552

(51) Int. Cl. G01N 33/52; C12Q 1/6834

(52) U.S. Cl. CPC G01N 33/521; C12Q 1/6834

(57) ABSTRACT

A kit for detecting a nucleic acid and a method for preparing the same are provided. The kit includes: a test area which has a lower surface formed as a concave curved surface and includes molecular beacons concentrated on a glass fiber network; a surrounding area which completely surrounds the test area; and a support area which surrounds the surrounding area, wherein the surrounding area includes a hydrophobic polymer on the glass fiber network, the test area does not include the hydrophobic polymer, and a glass fiber in the test area is functionalized.

### CROSS-REFERENCE TO RELATED APPLICATION

This application claims the benefit of Korean Patent Application No.10-2021-0088552, filed on Jul. 6, 2021 in the Korean Intellectual Property Office, the entire disclosures of which are incorporated herein by reference.

### BACKGROUND

### 1. Field of the Invention

The present disclosure relates to a kit for detecting nucleic acid and a method of preparing the same.

### 2. Discussion of Related Art

There are numerous methods of detecting nucleic acid. A general principle of detecting nucleic acid is that a detection substance capable of detecting nucleic acid is fixed onto a test area, a sample containing nucleic acid to be detected is added to the test area, and a reaction between the nucleic acid and the detection substance is determined on the basis of a presence or absence of luminescence or fluorescence. In recent years, molecular beacons which are fluorescent molecule labels have been used as the detection substance capable of detecting nucleic acid. However, when concentrating the molecular beacons, a degree of concentration of the molecular beacons should be high for the nucleic acid to be detected with a high degree of sensitivity.

Meanwhile, as devices for detecting a nucleic acid, most fiber-based detection devices are manufactured with a hydrophilic cellulose or nitrocellulose fiber.

However, the fiber-based nucleic acid detection devices that are currently commercialized have a problem that the amount of a nucleic acid in a sample should be high. Thus, in a case in which the amount of a nucleic acid in a sample is small, polymerase chain reaction (PCR) that replicates and amplifies a nucleic acid is essentially required. PCR cannot be performed on detection site in real time as necessary, causing an increase in detection time, and as a result, has a problem that it extends the time necessary for determining a presence or absence of a nucleic acid.

The related art of the present disclosure is disclosed in Korean Patent Registration No. 10-1768146.

### SUMMARY OF THE INVENTION

The present disclosure is directed to providing a nucleic acid detection kit which can provide a nucleic acid detection effect even when a relatively small number of molecular beacons is concentrated.

The present disclosure is also directed to providing a nucleic acid detection kit which can detect even an infinitesimal amount of a nucleic acid with a high degree of sensitivity, thus lowering a limit of nucleic acid detection, not requiring a PCR process for nucleic acid amplification, and enabling prompt, on-site diagnosis of a nucleic acid.

The present disclosure is also directed to providing a method of preparing a nucleic acid detection kit that allows the nucleic acid detection kit of the present disclosure to be prepared with a simple method, thus improving a process of the preparation.

One aspect of the present disclosure relates to a nucleic acid detection kit.

1. A nucleic acid detection kit includes: a test area which has a lower surface formed as a concave curved surface and includes molecular beacons concentrated on a glass fiber network; a surrounding area which completely surrounds the test area; and a support area which surrounds the surrounding area, wherein the surrounding area includes a hydrophobic polymer on the glass fiber network, the test area does not include the hydrophobic polymer, and a glass fiber in the test area is functionalized.

2. In 1, the surrounding area may have a property of blocking an aqueous solution.

3. In 1 and 2, the glass fiber in the test area may be functionalized as a functional group of which an outermost end is a maleimide group.

4. In 1 to 3, the hydrophobic polymer may be a biocompatible polymer.

Another aspect of the present disclosure relates to a method of preparing the nucleic acid detection kit of the present disclosure.

5. A method of preparing a nucleic acid detection kit, which is a method of preparing the nucleic acid detection kit of the present disclosure, includes: preparing a substrate that includes a glass fiber network that at least includes a functionalized glass fiber; forming the surrounding area on a portion of the substrate by using a surrounding area formation device; and preparing the test area by concentrating molecular beacons on a portion surrounded by the surrounding area, wherein the surrounding area formation device is hollow and extends from an upper surface to a lower surface, thus having an outer diameter and an inner diameter, a groove is formed along the outer diameter or the inner diameter between the outer diameter and the inner diameter on the lower surface, the groove is filled with the hydrophobic polymer, and forming the surrounding area includes pressing the surrounding area formation device into a portion where the surrounding area is desired to be formed and causing the hydrophobic polymer to be absorbed into the portion where the surrounding area is desired to be formed.

6. In 5, the groove may have a height larger than a cross-sectional width thereof, and the height of the groove may be in a range of 0.1 mm to 2 mm.

7. In 5 and 6, a thickness of the substrate may be thicker than a height of the groove.

The present disclosure can provide a nucleic acid detection kit which can provide a nucleic acid detection effect even when a relatively small number of molecular beacons is concentrated.

The present disclosure can provide a nucleic acid detection kit which can detect even an infinitesimal amount of a nucleic acid with a high degree of sensitivity, thus lowering the limit of nucleic acid detection, not requiring the PCR process for nucleic acid amplification, and enabling prompt, on-site diagnosis.

The present disclosure can provide a method of preparing a nucleic acid detection kit that allows the nucleic acid detection kit of the present disclosure to be prepared with a simple method, thus improving a process of the preparation.

### BRIEF DESCRIPTION OF THE DRAWINGS

The above and other objects, features and advantages of the present disclosure will become more apparent to those of ordinary skill in the art by describing exemplary embodiments thereof in detail with reference to the accompanying drawings, in which:

FIG. 1 shows a conceptual diagram (A) of a nucleic acid detection kit and a conceptual diagram (B) of operation when nucleic acid is injected into the nucleic acid detection kit according to one embodiment of the present disclosure;

FIG. 2 is a cross-sectional view of the nucleic acid detection kit that is taken along I-II of FIG. 1 according to one embodiment of the present disclosure;

FIG. 3 shows a method of functionalizing a surface of a glass fiber in the nucleic acid detection kit according to one embodiment of the present disclosure;

FIG. 4 shows a process of preparing the nucleic acid detection kit according to one embodiment of the present disclosure;

FIG. 5 shows a perspective view (A), a lateral view (B), a cross-sectional view (C), an upper plan view (D), and a lower plan view (E) of a surrounding area formation device in FIG. 4;

FIG. 6 sequentially illustrates an impregnation process of a hydrophobic polymer in a process of stamping the surrounding area formation device in FIG. 4;

FIG. 7 shows results showing fluid forms when a fluid is dripped in Example 1 ((a)), Comparative Example 1 ((b)), and Comparative Example 2 ((c));

FIG. 8 shows detection results of Comparative Example 3 ((a)) and Example 1 ((b)); and

FIG. 9 shows detection results of Example 1 ((a)) and Comparative Example 4 ((b)).

### DETAILED DESCRIPTION OF EXEMPLARY EMBODIMENTS

Embodiments of the present disclosure will be described in detail with reference to the accompanying drawings to allow those of ordinary skill in the art to which the present disclosure pertains to easily carry out the embodiments of the present disclosure. The present disclosure may be implemented in various different forms and is not limited to the embodiments described herein. In the drawings, parts unrelated to the description are omitted for clarity of the present disclosure, and the same or similar elements are denoted by the same reference numerals throughout. In the drawings, thicknesses, lengths, widths, or the like of the corresponding parts are drawn to arbitrary scale and do not limit the scope of the present disclosure.

A nucleic acid detection kit of the present disclosure includes a test area which is disposed on a support area and in which molecular beacons are concentrated. The molecular beacons are fluorescent molecule labels for detecting nucleic acids and detect nucleic acids that may undergo a complementary reaction in the test area. When nucleic acids that may react with the molecular beacons are added to the test area, the corresponding nucleic acids and the molecular beacons react, and the presence of nucleic acids to be detected is detected by fluorescence or the like.

In the nucleic acid detection kit of the present disclosure, the test area has a lower surface formed as a concave curved surface, and the nucleic acid detection kit includes a surrounding area that completely surrounds the test area. Here, being “completely surrounded” means that the entire outer side surface of the test area is surrounded by the surrounding area.

The surrounding area includes a hydrophobic polymer and has a three-dimensional hydrophobic fiber pattern that may form the test area. In this way, when it is attempted to concentrate the molecular beacons in the test area, a nucleic acid detection effect can be provided even when a relatively small number of molecular beacons is concentrated as compared to a kit in which the surrounding area is not formed. Also, even when a small amount of the nucleic acid is included in a sample to be measured, the nucleic acid cannot escape out of the test area, and thus even the small amount of the nucleic acid can be detected as compared to a kit in which the surrounding area is not formed. In this way, a limit of nucleic acid detection can be lowered, a PCR process for nucleic acid amplification is not required, and prompt, on-site diagnosis is possible. On the other hand, the test area does not include the hydrophobic polymer.

In one embodiment, the surrounding area may have a property of blocking an aqueous solution. In this way, the molecular beacons can be concentrated in a smaller number in the test area, and by preventing a nucleic acid-containing sample to be measured from escaping out of the test area, even a small amount of the nucleic acid can be detected without PCR.

In the nucleic acid detection kit of the present disclosure, the test area includes a glass fiber network, and the molecular beacons are concentrated on the glass fiber network. The glass fiber has a length longer than a diameter thereof and has a large surface area per unit volume and thus can concentrate a larger number of molecular beacons. Also, due to the fiber itself not having fluorescence, the glass fiber can prevent interference with fluorescence that occurs when the molecular beacons detect target nucleic acid.

The glass fiber may be functionalized. Unfunctionalized glass fibers in the test area may cause damage to the structure of the molecular beacons due to interacting with the molecular beacons when the molecular beacons are concentrated, and thus a false positive reaction may occur. When a surface of the glass fiber is functionalized, structural stability of the molecular beacons can be secured and thus reliability of the nucleic acid detection can be improved. Also, the functionalized surface of the glass fiber may allow the molecular beacons to be stably present in the glass fiber.

In one embodiment, the surface of the glass fiber may be functionalized as a functional group of which an outermost end is a maleimide group. That is, the maleimide group may be exposed at the outermost surface of the glass fiber. Unfunctionalized glass fibers in the test area may cause damage to the structure of the molecular beacons due to interacting with the molecular beacons when the molecular beacons are concentrated, and thus a false positive reaction may occur. When the surface of the glass fiber is functionalized as a functional group of which an outermost end is a maleimide group, structural stability of the molecular beacons can be secured and thus reliability of the nucleic acid detection can be improved.

In the nucleic acid detection kit of the present disclosure, the hydrophobic polymer is a biocompatible polymer. The biocompatible polymer may provide an effect of allowing the molecular beacons to be stably concentrated. A silicon-based polymer may be one type of biocompatible polymer. In one embodiment, the silicon-based polymer may include polydimethylsiloxane. Polydimethylsiloxane may, when forming the surrounding area which will be described below, facilitate the formation of the surrounding area.

The nucleic acid detection kit of the present disclosure allows the nucleic acid to be detected with a low limit of detection when nucleic acids of different concentrations are dripped and whether fluorescence occurs is determined. Also, the nucleic acid detection kit of the present disclosure allows nucleic acid to be detected without a PCR process even when the amount of nucleic acid in a sample is small. Fluorescence may be detected using fluorescence detectors such as multi pixel photon counters (MPPCs).

A method of preparing the nucleic acid detection kit of the present disclosure includes preparing a support area that includes a glass fiber network, forming the surrounding area on a portion of the support area by using a surrounding area formation device, and preparing the test area by concentrating molecular beacons on a portion surrounded by the surrounding area, wherein the surrounding area formation device is hollow and extends from an upper surface to a lower surface, thus having an outer diameter and an inner diameter, a groove is formed along the outer diameter or the inner diameter between the outer diameter and the inner diameter on the lower surface, the groove is filled with the hydrophobic polymer, and forming the surrounding area includes pressing the surrounding area formation device into a portion where the surrounding area is desired to be formed and causing the hydrophobic polymer to be absorbed into the portion where the surrounding area is desired to be formed.

According to the method of preparing the nucleic acid detection kit of the present disclosure, by using the surrounding area formation device which will be described below, filling the groove of the surrounding area formation device with the hydrophobic polymer, and simply pressing the surrounding area formation device, it is possible to form the surrounding area in an easy way.

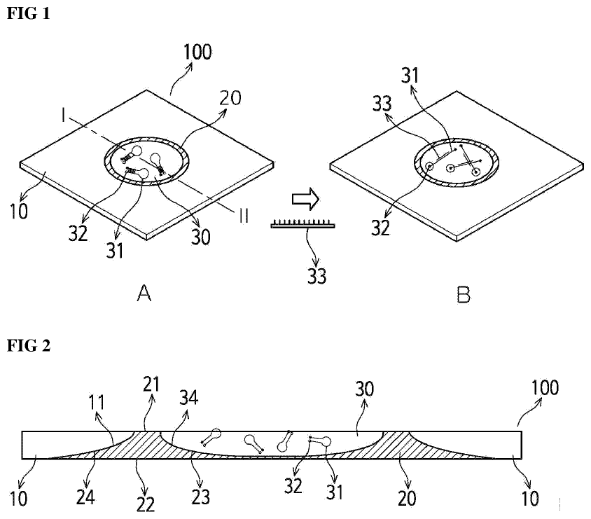

Hereinafter, a nucleic acid detection kit according to one embodiment of the present disclosure will be described with reference to FIGS. 1, 2, and 3.

Referring to FIG. 1A, a nucleic acid detection kit 100 may include a support area 10, a test area 30, and a surrounding area 20.

The support area 10, the test area 30, and the surrounding area 20 are integrally formed. Being “integrally formed” means that the support area, the test area, and the surrounding area are not separated from each other.

In one embodiment, the support area, the test area, and the surrounding area may be connected to each other by a glass fiber network (not illustrated).

In one embodiment, the kit including the support area, the test area, and the surrounding area is prepared using paper which is a hydrophilic porous medium having a high porosity. A glass fiber is one type of the hydrophilic porous medium having a high porosity.

The porosity of the glass fiber is in a range of 0.75 to 0.90 and can be said to be relatively higher than a porosity of paper made of different fibers. A cross-sectional diameter of the glass fiber may be in a range of 1 μm to 25 μm or may be in a range of 5 μm to 10 μm. In such ranges, the glass fiber can be used for the nucleic acid detection kit of the present disclosure, and since a surface area per unit volume is large, a larger number of molecular beacons can be concentrated in the test area.

A surface of the glass fiber may be functionalized as a functional group of which an outermost end is a maleimide group so that the maleimide group is exposed at the outermost surface of the glass fiber. The glass fiber included in one or more of the test area, the surrounding area, and the support area may be functionalized as above.

In one embodiment, the outermost surface of the glass fiber may be functionalized with a moiety represented by Chemical Formula 1 below:

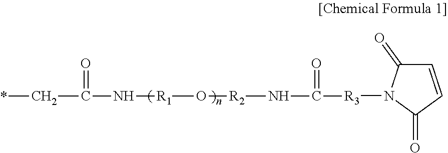

(In Chemical Formula 1 above,

* represents a portion connected to oxygen of the surface of the glass fiber,

R₁, R₂, and and R₃ each independently represent an alkylene group having a carbon number of 1 to 10, and

n represents an integer in a range of 20 to 100).

In one embodiment, the surface of the glass fiber may be functionalized according to Chemical Formula 2 below:

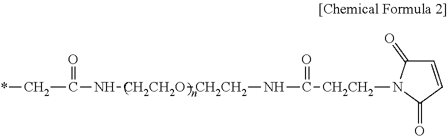

(In Chemical Formula 2 above,

* represents a portion connected to oxygen of the surface of the glass fiber, and

n represents an integer in a range of 20 to 100).

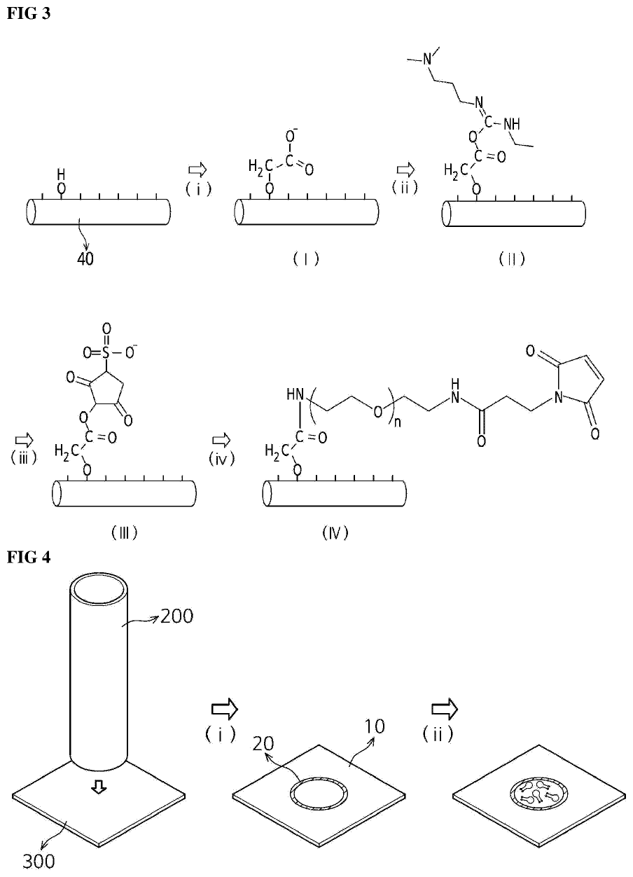

A method of functionalizing the glass fiber as a functional group of which an outermost end is a maleimide group will be described with reference to FIG. 3.

Referring to FIG. 3, in step (i), a glass fiber 40 is treated with sodium chloroacetate and sodium hydroxide to prepare a glass fiber I whose surface is functionalized. The content and reaction conditions of each of sodium chloroacetate and sodium hydroxide may be controlled according to glass fiber content or the like.

In step (ii), the glass fiber I whose surface is functionalized is caused to react with 1-ethyl-3-(3-dimethylaminopropyl)carbodiimide (EDC) to prepare a glass fiber II whose surface is functionalized. The content and reaction conditions of EDC may be controlled according to glass fiber content or the like.

In step (iii), the glass fiber II whose surface is functionalized is caused to react with N-hydroxysulfosuccinimide(Sulfo NHS) to prepare a glass fiber III whose surface is functionalized. The content and reaction conditions of N-hydroxysulfosuccinimide may be controlled according to glass fiber content or the like.

In step (iv), the glass fiber III whose surface is functionalized is caused to react with primary amine to prepare a glass fiber IV whose surface is functionalized. The content and reaction conditions of primary amine may be controlled according to glass fiber content or the like. The primary amine may include one or more of maleimide group-containing polyethylene glycol (PEG)-based amines. In the glass fiber IV, n may represent an integer in a range of 20 to 100. For example, the primary amine may be

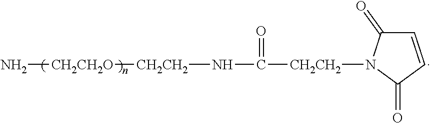

Referring again to FIG. 1, the test area 30 includes molecular beacons concentrated on the glass fiber network. The molecular beacons each include a fluorescent label 32 and a nucleic acid detection probe 31. The molecular beacons may be selectively used according to the type of nucleic acid to be detected, a site that specifically bonds to the nucleic acid to be detected, or the like.

Referring to FIG. 1B, when nucleic acid 33 is added to the test area 30, the nucleic acid and the molecular beacons specifically bond and cause fluoresence to be exhibited. In this way, presence of the nucleic acid can be recognized.

FIG. 2 is a cross-sectional view taken along I-II of FIG. 1.

Referring to FIG. 2, the test area 30 has a lower surface formed as a concave curved surface 34 and is formed in the shape of a bowl, and the surrounding area 20 is formed to completely surround the test area 30. The test area 30 and the surrounding area 20 are formed to be in direct contact with each other, and the surrounding area 20 includes a hydrophobic polymer and has an effect of blocking an aqueous solution. On the other hand, the test area 30 does not include a hydrophobic polymer. In this way, the nucleic acid detection kit of the present disclosure can provide the above-described effects. The lower surface, which is the concave curved surface, of the test area 30 and the surrounding area 20 may be implemented by a method of preparing the nucleic acid detection kit of the present disclosure which will be described below.

In one embodiment, the maximum thickness of the test area 30 may be smaller than the maximum thickness of the nucleic acid detection kit 100, and a part of the surrounding area 20 may be disposed on the lower surface of the test area 30. By the part of the surrounding area 20 being disposed on the lower surface of the test area 30, the above-described effects of the present disclosure can be obtained in a process in which the molecular beacons are concentrated and a process in which nucleic acid to be detected is added.

For example, the maximum thickness of the test area 30 may be in a range of 80 μm to 800 μm or may be in a range of 240 μm to 560 μm. For example, the maximum thickness of the surrounding area 20 may be in a range of 100 μm to 1,000 μm or may be in a range of 300 μm to 700 μm. For example, the thickness of the nucleic acid detection kit 100 may be in a range of 100 μm to 1,000 μm or may be in a range of 300 μm to 700 μm. In such ranges, the test area 30 and the surrounding area 20 can be used for the nucleic acid detection kit of the present disclosure.

In one embodiment, the surrounding area 20 may have an uppermost surface 21 formed as a flat surface, a lowermost surface 22 formed as a flat surface, and one surface 23 connected to the uppermost surface 21 and formed to come in direct contact with the concave curved surface 34, which is the lower surface of the test area 30. Another surface 24 connected to the uppermost surface 21 may come in direct contact with a concave curved surface 11, which is a lower surface of the support area 10.

In one embodiment, an upper surface of the test area 30 may have a circular shape, an elliptical shape, an oval shape, a polygonal shape, an amorphous shape, or the like. Here, the upper surface of the test area 30 is completely surrounded by the surrounding area 20.

The upper surface of the test area 30 may have the longest diameter in a range of 0.8 mm to 8 mm, and the longest diameter of an upper surface of the surrounding area 20 may be larger than the longest diameter of the upper surface of the test area 30 and may be in a range of 2 mm to 11 mm.

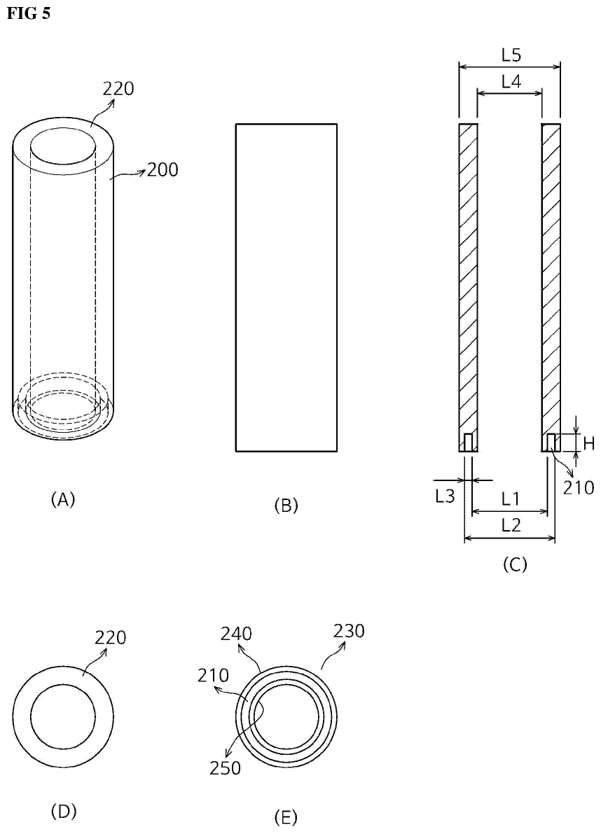

Hereinafter, a method of preparing a nucleic acid detection kit according to one embodiment of the present disclosure will be described with reference to FIGS. 4, 5, and 6.

Referring to FIG. 4, the method of preparing the nucleic acid detection kit includes step (i) and step (ii).

Referring to step (i), a substrate 300 including a glass fiber network is prepared, a surrounding area formation device 200 is pressed from top to bottom on one portion of the substrate 300, and then drying is performed to form the surrounding area 20 and the support area 10 on the one portion of the substrate 300.

The substrate 300 including the glass fiber network later becomes a material for forming the support area, the test area, and the surrounding area of the nucleic acid detection kit of the present disclosure. The glass fiber network may be functionalized according to the above-described method before the surrounding area formation device 200 is pressed from top to bottom.

The surrounding area formation device 200 has a shape that is hollow and extends from an upper surface to a lower surface, thus having an outer diameter and an inner diameter. A groove is formed along the outer diameter or the inner diameter between the outer diameter and the inner diameter on the lower surface of the surrounding area formation device, and the groove is filled with a hydrophobic polymer. The surrounding area formation device will be described in more detail with reference to FIG. 5.

Referring to FIG. 5, the surrounding area formation device 200 has a shape that is hollow and extends from an upper surface 220 to a lower surface 230, thus having an outer diameter 240 and an inner diameter 250. A groove 210 is formed along the outer diameter 240 or the inner diameter 250 between the outer diameter 240 and the inner diameter 250 on the lower surface 230 of the surrounding area formation device. The groove 210 is formed in a concave shape.

A height H of the groove 210 may be in a range of 0.1 mm to 2 mm or may be in a range of 0.5 mm to 1 mm. A width L3 of the groove 210 may be in a range of 0.1 mm to 1 mm or may be in a range of 0.3 mm to 0.7 mm. In such ranges, the formation of the surrounding area of the present disclosure may be facilitated.

A thickness of the substrate may be thicker than the height of the groove.

A shortest diameter L1 of the groove 210 may be in a range of 1 mm to 8 mm or may be in a range of 3 mm to 6 mm. A longest diameter L2 of the groove 210 may be in a range of 2 mm to 9 mm or may be in a range of 4 mm to 7 mm. In such ranges, the formation of the surrounding area of the present disclosure may be facilitated.

A diameter L5 of the outer diameter 240 may be in a range of 3 mm to 11 mm or may be in a range of 5 mm to 9 mm. A diameter L4 of the inner diameter 250 may be in a range of 0.8 mm to 7.8 mm or may be in a range of 2 mm to 5 mm. In such ranges, the formation of the surrounding area of the present disclosure may be facilitated.

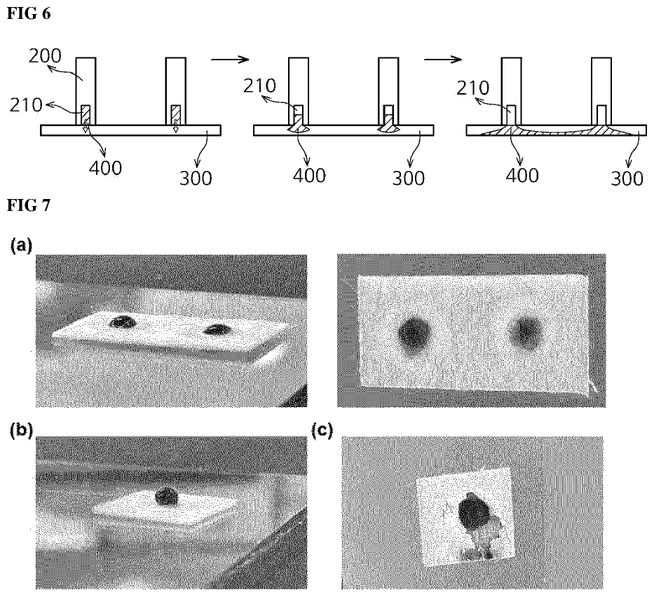

The groove 210 is filled with a hydrophobic polymer. When the surrounding area formation device 200 is pressed from top to bottom, the hydrophobic polymer filled in the groove is impregnated into the substrate and forms the surrounding area. The hydrophobic polymer is the same as that described above. This will be described with reference to FIG. 6.

Referring to FIG. 6, a hydrophobic polymer 400 in the surrounding area formation device 200 moves in directions indicated by arrows due to gravity and is impregnated into the substrate 300. The hydrophobic polymer is impregnated into a portion of the substrate on which the test area is desired to be formed, and thus the surrounding area is formed. The type of hydrophobic polymer, the porosity of the glass fiber network, specific specifications of a stamp-type structure, the thickness of the support area, and the like may be controlled to form the surrounding area of the present disclosure.

After the hydrophobic polymer is completely impregnated, drying may be performed at a temperature in a range of 70° C. to 120° C., preferably at a temperature of 80° C., for 1 to 4 hours, preferably for 2 hours, to completely form the surrounding area.

The surrounding area formation device has a stamp-type structure and may be produced using a general method known to those of ordinary skill in the art. For example, the surrounding area formation device may be produced using a 3D printer, but the method of producing the surrounding area formation device is not limited thereto.

Referring again to FIG. 4, in step (ii), the test area is prepared by concentrating molecular beacons on the portion surrounded by the surrounding area. This may be performed by dropping the molecular beacons onto the portion surrounded by the surrounding area and then drying.

Hereinafter, the configurations and actions of the present disclosure will be described in more detail using exemplary examples of the present disclosure. However, the following examples are only presented as exemplary examples of the present disclosure, and the present disclosure should not be construed as being limited by the examples in any way.

### EXAMPLE 1

Glass fiber paper including a glass fiber functionalized using the method illustrated in FIG. 3 was prepared. A stamp-type structure having a shape shown in FIG. 5 and having an outer diameter of 6 mm, an inner diameter of 4 mm, and a groove having a height of 1 mm, the longest diameter of 5.5 mm, and the shortest diameter of 4.5 mm was produced using a 3D printer to produce a surrounding area formation device. The groove of the surrounding area formation device was filled with polydimethylsiloxane. The produced surrounding area formation device was stamped on a portion of the glass fiber paper on which a surrounding area was desired to be formed. Then, the glass fiber paper was kept in an oven at a temperature of 80° C. for 2 hours to completely fix polydimethylsiloxane thereon and form the surrounding area. Molecular beacons (Preparer: Integrated DNA Technologies) were dropped onto a portion surrounded by the surrounding area and dried to prepare a test area of a nucleic acid detection kit. A cross-section of the prepared nucleic acid detection kit is shown in FIG. 2.

### COMPARATIVE EXAMPLE 1

A kit was prepared in the same way as in Example 1 except that not only the surrounding area but also a test area was made of polydimethylsiloxane in Example 1.

### COMPARATIVE EXAMPLE 2

A kit was prepared in the same way as in Example 1 except that the surrounding area was not disposed on a lower surface of the test area and thus the surrounding area did not completely surround the test area in Example 1.

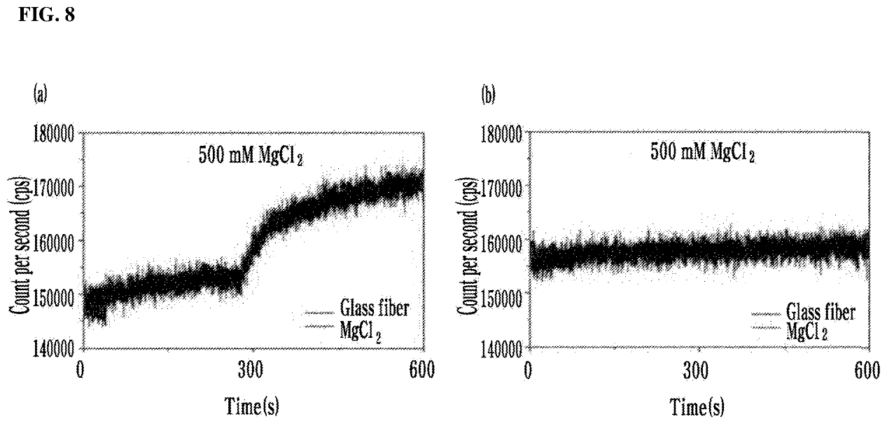

A fluid (a solution obtained by mixing red ink in deionized (DI) water) was dripped onto the test areas of the kits prepared in Example 1, Comparative Example 1, and Comparative Example 2. Results thereof are shown in FIG. 7.

As shown in FIG. 7, it can be seen that in Example 1, the fluid was trapped in the test area, and a certain amount of the fluid was absorbed. On the other hand, it can be seen that in Comparative Example 1, the fluid was not absorbed at all due to the test area being hydrophobic. In Comparative Example 2, the fluid completely escaped through the lower surface of the kit, and measurement itself was not possible.

### COMPARATIVE EXAMPLE 3

A kit was prepared in the same way as in Example 1 except that the glass fiber was not functionalized in Example 1.

500 mM MgCl₂ was dropped onto the test areas of the kits prepared in Example 1 and Comparative Example 3, and whether fluorescence occurred was checked. Results thereof are shown in FIG. 8.

As shown in FIG. 8, a false positive reaction occurred in Comparative Example 3 in which the glass fiber was not functionalized. On the other hand, a reaction did not occur at all in Example 1 in which the glass fiber was functionalized.

### COMPARATIVE EXAMPLE 4

A lateral flow analysis device (lateral flow strip, self-produced) was used.

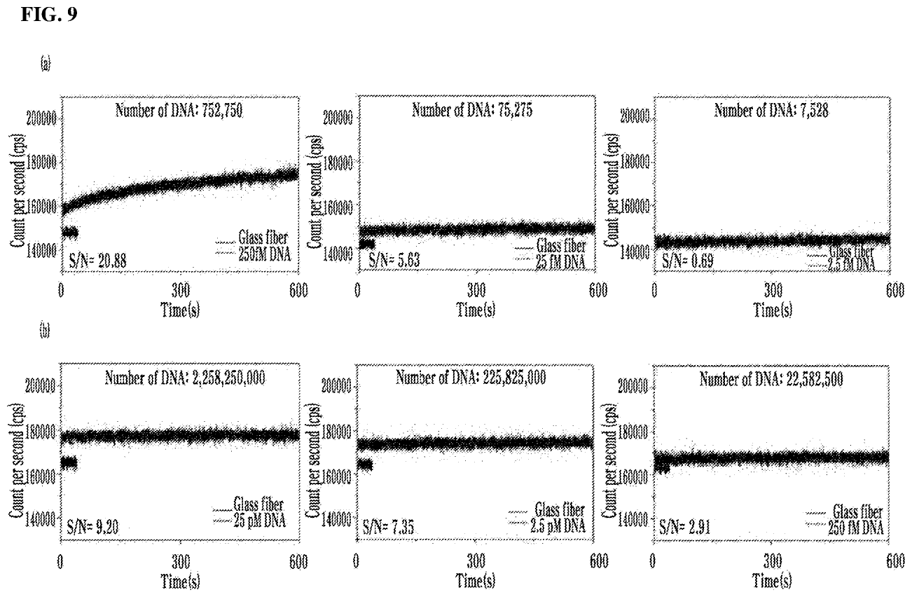

Samples containing nucleic acid in the same concentration were prepared. The nucleic acid contained in the samples exhibited fluorescence upon a reaction with the molecular beacons included in the test area. 5 μl of the sample was added in Example 1, 150 μl of the sample was added to the conventional lateral flow analysis device, and whether fluorescence occurred was checked. Results thereof are shown in FIG. 9.

As shown in FIG. 9, it can be seen that in Example 1, a limit of detection was significantly lowered as compared to Comparative Example 4.

Simple modifications or changes to the present disclosure may be easily made by those of ordinary skill in the art, and all of such modifications or changes should be construed as belonging to the scope of the present disclosure.

What is claimed is:

1. A nucleic acid detection kit comprising:
a test area which has a lower surface formed as a concave curved surface and includes molecular beacons concentrated on a glass fiber network;
a surrounding area which completely surrounds the test area; and
a support area which surrounds the surrounding area,
wherein the surrounding area includes a hydrophobic polymer on the glass fiber network,
the test area does not include the hydrophobic polymer, and
a glass fiber in the test area is functionalized.

2. The nucleic acid detection kit of claim 1, wherein the surrounding area has a property of blocking an aqueous solution.

3. The nucleic acid detection kit of claim 1, wherein the glass fiber in the test area is functionalized as a functional group of which an outermost end is a maleimide group.

4. The nucleic acid detection kit of claim 1, wherein the hydrophobic polymer is a biocompatible polymer.

5. A method of preparing the nucleic acid detection kit of claim 1, the method comprising:
preparing a substrate that includes a glass fiber network that at least includes a functionalized glass fiber;
forming the surrounding area on a portion of the substrate by using a surrounding area formation device; and
preparing the test area by concentrating molecular beacons on a portion surrounded by the surrounding area,
wherein the surrounding area formation device is hollow and extends from an upper surface to a lower surface, thus having an outer diameter and an inner diameter, a groove is formed along the outer diameter or the inner diameter between the outer diameter and the inner diameter on the lower surface, and the groove is filled with the hydrophobic polymer, and
forming the surrounding area includes pressing the surrounding area formation device toward a portion where the surrounding area is desired to be formed and causing the hydrophobic polymer to be absorbed into the portion where the surrounding area is desired to be formed.

6. The method of claim 5, wherein the groove has a height larger than a cross-sectional width thereof, and the height of the groove is in a range of 0.1 mm to 2 mm.

7. The method of claim 5, wherein a thickness of the substrate is thicker than a height of the groove.
